(19) United States
(12) Patent Application Publication
(10) Pub. No.: US 2026/0345303 A1
(43) Pub. Date: Nov. 5, 2026

(54) Interdental Cleaner with Sensor

(71) Applicant: Trustees of Tufts College, Medford, MA (US)

(72) Inventors: Sameer Sonkusale, Lincoln, MA (US); Atul Sharma, Medford, MA (US)

(21) Appl. No.: 19/131,068

(22) PCT Filed: Nov. 22, 2023
(86) PCT No.: PCT/US2023/080912

§ 371 Date: May 19, 2025

Related U.S. Application Data
(60) Provisional application No. 63/427,229, filed on Nov. 22, 2022.

(51) Int. Cl. A61C 15/04; A61C 19/04

(52) U.S. Cl. CPC A61C 15/04; A61C 19/04; A61C 2201/00

(57) ABSTRACT

An interdental cleaner includes a handle, wicking material, a sensor, and sensing circuitry. The sensor and the sensing circuitry arc disposed in the handle and the wicking material includes a first length and a second length. The first length is external to the handle and the second length extends into the handle to make contact with the sensor. Exposure of the first length to saliva causes capillary transport of the saliva towards the second length. The sensor includes a sensing element that is functionalized to selectively bind to the analyte in the saliva.

### RELATED APPLICATIONS

This application claims the benefit of the Nov. 22, 2022 priority date of U.S. Provisional Application 63/427,229, the content of which is herein incorporated by reference.

### STATEMENT OF GOVERNMENT RIGHTS

This invention was made with government support under grant 1931978 awarded by the National Science Foundation. The government has certain rights in the invention.

### FIELD OF INVENTION

This invention pertains to sensors and in particular to sensors that sense substances in saliva.

### BACKGROUND

Biomarkers found in a person's bodily fluids provide considerable information on the health of that person. It is for this reason that an annual physical examination almost invariably includes drawing blood or collecting a urine sample.

Saliva, too, contains many biomarkers of interest. Such biomarkers are useful for diagnosis of dental disease. In addition, certain systemic diseases are made manifest through biomarkers in saliva.

Despite its usefulness as a source of biomarkers, the collection of saliva is not nearly as easy as collection of blood or urine. The supply of saliva is meager to begin with. And rather than collecting in bodily cavities or circulating through the body, saliva is swallowed soon after being produced.

Known ways of collecting saliva include placing a sensor in a person's mouth for extended periods. An example is a sensor integrated into a dental appliance, such as a mouthguard. However, this is often an inconvenience to the person who has to wear it.

### SUMMARY

The invention integrates the process of saliva analysis into customary dental hygiene activities. In the preferred embodiment, the analysis of saliva begins with the act of using a wicking material, such as floss, the clean the spaces surrounding one's teeth.

Embodiments include those in which the coating comprises gold as well as those in which it comprises silver, zinc, platinum, or carbon.

A variety of biomarkers are detectable using the apparatus. These include cortisol, epinephrine, interleukin-6, Neuropeptide-Y, TNF-alpha, MMP-9, MMP-12, interleukin-1, interleukin-8, procalcitonin, and the like. These can be sensed by exposure to an appropriate antibody.

In some embodiments, sensing is carried out electrochemically. In others, sensing is carried out optically. Among these are embodiments that rely on colorimetric and fluorometric detection of the markers in saliva directly on the floss.

In one aspect, the invention features an interdental cleaner having a frame, a handle, floss, an electrode array, and sensing circuitry. The electrode array and the sensing circuitry are disposed in the handle. The floss comprises a first length, which extends across the frame, and a second portion, which extends and into the handle to make contact with electrodes in the electrode array. Exposure of the first length to saliva causes the saliva to be transported towards the second length by capillary action. The sensing circuitry is configured to sense an analyte in the saliva.

Among the embodiments are those that also include an output device that provides information indicative of the analyte. Examples of such an output device include a display disposed on the handle and in communication with the sensing circuitry. Other examples include a wireless communication module that outputs the information to another device, such as a smartphone or a reader.

Embodiments further include a reader that receives the handle and reads information about the analyte from the sensing circuitry in the handle.

In some embodiments, the electrode array comprises a reference electrode, a counter electrode, and a working electrode.

In other embodiments, the electrode array comprises an electrode made from a material cither carbon, graphite, or gold and an electrode that is made from screen-printed ink that comprises either silver, silver chloride, or platinum.

In another aspect, the invention features an interdental cleaner having a handle, wicking material, a sensor, and sensing circuitry. The sensor and the sensing circuitry are disposed in the handle and the wicking material includes a first length and a second length. The first length is external to the handle and the second length extends into the handle to make contact with the sensor. Exposure of the first length to saliva causes capillary transport of the saliva towards the second length. The sensor includes a sensing element that is functionalized to selectively bind to the analyte in the saliva.

Embodiments include those in which the first length extends across arms of a frame and those in which it comprises a stiff wicking material that extends away from the handle.

Further embodiments include a display disposed on the handle and in communication with the sensing circuitry. The display displays information that it receives from the sensing circuitry. This information is indicative of the analyte.

Still other embodiments include a reader that couples to the handle and reads information about the analyte from the sensing circuitry in the handle.

Embodiments further include those in which the sensor includes a reference electrode, a counter electrode, and a working electrode and those in which the sensor includes an electrochemical sensor.

Still other embodiments include those in which the sensor includes an optical sensor. Among these are embodiments in which the sensor includes a radiation source, a radiation detector, and a fiber extending therebetween. In such embodiments, the fiber is coupled to a sensor element having an optical property that changes in response to presence of the analyte. Among these embodiments are those in which the sensor element selectively binds to the analyte. Embodiments that include an optical sensor include those in which the detector senses resonance of surface plasmons on the sensor element and those in which the detector includes a spectrometer that senses a lengthening of wavelength of electromagnetic radiation as a result of a loss of energy in the electromagnetic radiation due to interaction thereof with the sensor element.

In some embodiments, the sensor element is one of a plurality of sensor elements of the sensor, each of which has been functionalized for a corresponding analyte in the saliva, wherein the circuitry includes a multiplexer, a controller, and an analyzer, and wherein the multiplexer receives a selection signal from the controller and, in response, provides a signal from a corresponding one of the sensor elements to the analyzer.

Further embodiments include those in which the sensing element is functionalized by an antibody that selectively binds to the analyte, those in which the sensing element is functionalized by an aptamer that selectively binds to the analyte, those in which the sensing element is functionalized by a molecularly-imprinted polymer that selectively binds to the analyte, those in which the sensing element is functionalized by an enzyme that selectively binds to the analyte, and those in which the sensing element is functionalized by a nanobody that selectively binds to the analyte.

Embodiments further include those in which the sensor includes an electrode that is made from a material selected from carbon, graphite, or gold and an electrode that is made from screen-printed ink that includes silver, silver chloride, or platinum.

In still other embodiments, the first length of the wicking material includes dental floss and the second length includes a microfluidic channel coupled to the first length.

In another aspect, the invention features a method that includes exposing a first length of a wicking material of an interdental cleaner to saliva, thereby causing saliva to flow by capillary action towards a second length of the wicking material, the second length being internal to a handle of the interdental cleaner, The method continues at a sensor in the handle with sensing an interaction of an analyte in the saliva with a functionalized sensor element of the sensor after which information indicative of the interaction is provided to a user.

These and other features of the invention will be apparent from the following detailed description and the accompanying figures, in which:

### BRIEF DESCRIPTION OF THE FIGURES

FIG. 1 shows an interdental cleaner,

FIG. 2 shows details of the interdental cleaner shown in FIG. 1,

FIG. 3 shows a power supply for charging the interdental cleaner of FIG. 1,

FIG. 4 shows circuitry within the interdental cleaner of FIG. 1;

FIG. 5 shows an embodiment of the interdental cleaner of FIG. 1 that relies on optical sensing;

FIG. 6 shows an alternative embodiment of the interdental cleaner in which the arms shown in FIG. 1 have been omitted; and

FIG. 7 shows steps in the manufacture of the interdental cleaner of FIG. 1.

### DETAILED DESCRIPTION

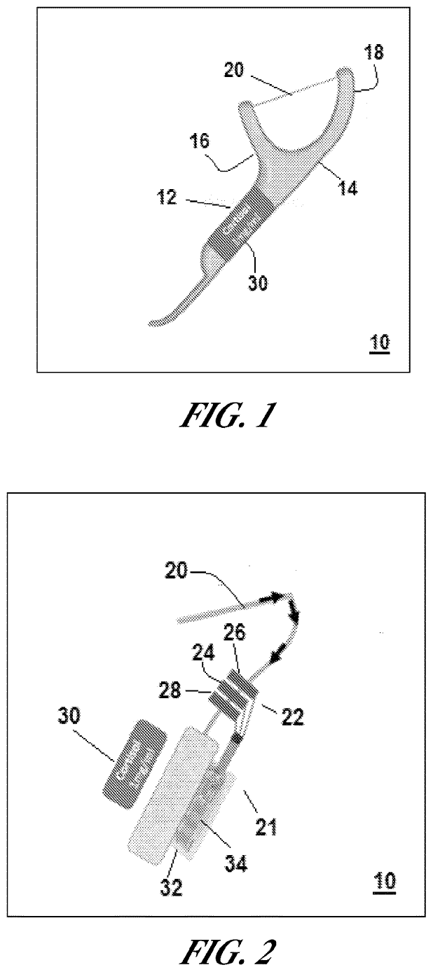

FIG. 1 shows an interdental cleaner 10. The interdental cleaner 10 is a unitary structure having a proximal end that forms a handle 12 and a distal end that forms a frame 14. The frame 14 includes first and second arms 16, 18. A first length of wicking material 20 spans a gap between the tips of the first and second arms 16, 18.

In preferred embodiments, the wicking material 20 is floss or a textile thread. Other useful materials for the wicking material 20 include nylon, polyamide, and polytetrafluoroethylene.

For ease of manufacturing, it is sometimes useful to make the wicking material 20 from separate lengths that are coupled together. In such cases, the second length is formed by a microfluidic thread that permits capillary flow to continue past the first length.

As one uses the interdental cleaner 10 to clean between one's teeth, the wicking material 20 draws saliva and guides it towards the handle 12 as a result of capillary action. In the process, the wicking material 20 also filters large particles and even certain large proteins. As a result, the saliva that reaches the handle 12 is relatively free of such impurities.

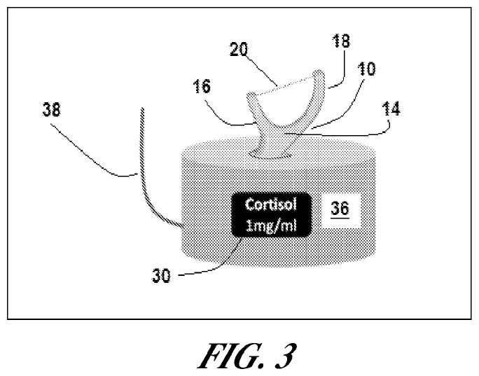

As shown in FIG. 2, a second length of the wicking material 20 extends proximally into the handle 12 where it encounters a sensor 22 having one or more sensor elements 24, 26. In the illustrated embodiment, the wicking material 20 encounters a sensor 22. In the illustrated embodiment, the sensor 22 is an electrochemical sensor in which the sensor elements 24, 26, 28 comprise first, second, and third electrodes.

An advantage of the configuration shown is that the handle 12 protects the second length of the wicking material 20, which is where actual sensing takes place, from abrasion and fouling. Another advantage is that the continuous flow of capillary action ensures that fresh saliva constantly flows past the sensor 22. As a result, the integrated volume of saliva that the sensor 22 samples is greater than its small spatial extent would suggest. This increases the probability that a target analyte will eventually reach the sensor 22 and thus promotes the sensor's ability to reliably sense a target analyte even at low concentrations thereof.

The first sensor element 24 is typically an electrode that has been screen printed using an ink that comprises substances such as silver, silver chloride ink, or platinum. Embodiments include those in which the first sensor element 24 is screen printed on a plastic sheet as well as those in which they are made directly on the wicking material 20 itself. This first sensor element 24 functions as a reference electrode. The second and third sensor elements 26, 28 are counter and working electrodes, respectively.

Suitable materials for the second or third sensor elements 26, 28 include carbon, graphite, gold, and any other material that avoids reacting with the analyte of interest.

To promote interaction with the analyte of interest, it is useful to functionalize a sensor element 26, 28. This is carried out by incorporating therein a substance that selectively binds to the analyte.

Examples of substances that are useful for functionalizing a sensor element 26, 28 include antibody or aptamer molecules or a polymer that has been placed on the polymer by molecular imprinting thereof. In a particular embodiment, the analyte is cortisol and the sensor element is functionalized with an antibody, a molecularly-imprinted polymer, or an aptamer that selectively binds to cortisol.

The use of a molecularly-imprinted polymer is particularly advantageous for its specificity and selectivity, its improved shelf life, its case of fabrication and low cost, and its tunability and the ease with which it can be chemically modified. In addition, there is no animal testing or in-vivo testing required. Such polymers are synthetic analogs to natural antibodies and thus form a complex using a “lock and key” mechanism to selectively bind to a molecule of the analyte.

As the saliva flows past the sensor 22, molecules of the analyte eventually interact with the functionalized sensor element 26, 28. The complexes that form as a result change a property of the functionalized sensor element 26, 28. The change provides the basis for a signal indicative of the presence of the analyte, which in turn activates a display 30, such as a liquid-crystal display, on the handle 12.

A printed-circuit board 32 within the handle 12 comprises circuitry 34 formed thereon. Embodiments include those in which the printed-circuit board 32 is rigid and those in which it is flexible. The circuitry 34 is configured to measure interaction of the analyte with the sensor 22 and to communicate the result beyond the interdental cleaner 10.

Embodiments include those in which the circuitry 34 measures the extent of such interaction by impedance spectroscopy, amperometry, pulse voltammetry, or any other electrochemical approach.

Also among the embodiments are those in which the circuitry 34 measures interactions with electromagnetic waves, such as by detecting plasmon resonance, by Raman spectroscopy, or color changes, for example with a colorimetric sensor. These embodiments include those that rely on observing interactions with electromagnetic waves in the visible spectrum, the microwave spectrum, and the infrared spectrum. Accordingly, as used herein, “optical” is not limited to visible light.

Moreover, the foregoing sensing modalities are not mutually exclusive. Accordingly, some embodiments are dual mode or multimodal. Among these are embodiments in which the sensor 22 includes both an electrochemical sensing element and an optical sensing element.

In some embodiments, the circuitry 34 provides a signal indicative of a biomarker to a drug-delivery that then administers a drug in response to a change in a biomarker level.

Still other embodiments include a pouch for collecting volatile organic compounds present in the saliva for analysis in a laboratory.

Particular embodiments also include a wireless communication module embedded within the handle 12. Examples of wireless communication include communication via a short-range network such as “BLUETOOTH” and communication via RFID. To save power, it is useful for the wireless communication module to be turned on only when a read-out is required. In such cases, information is relayed directly to a suitable device, such as a smartphone, thereby obviating the need for a display 30 on the handle 12. Some embodiments include a battery within the interdental cleaner 10.

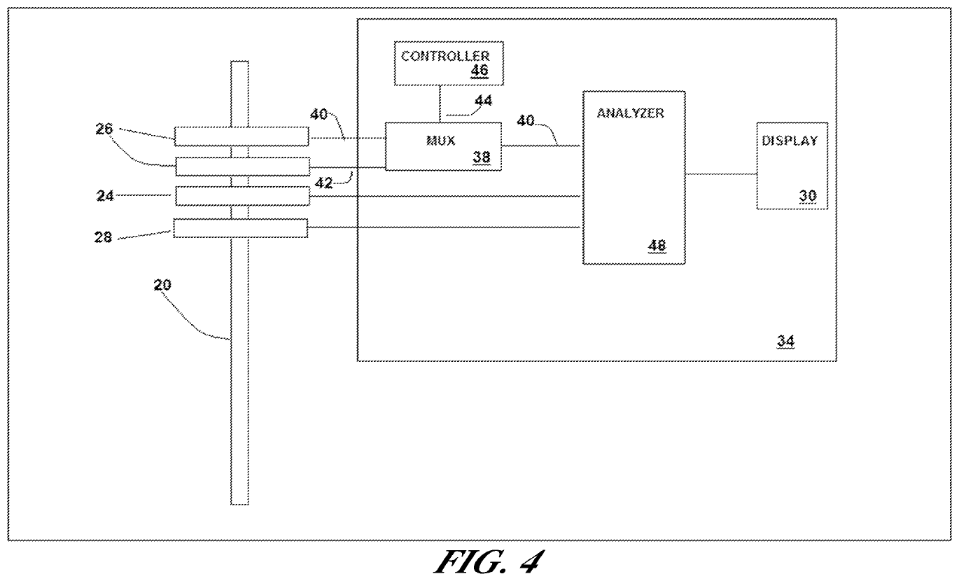

Referring to FIG. 3, some embodiments include a reading module 36 into which the handle 12 is inserted. The reading module 36 wirelessly reads the state of the sensor 22. It does so by using a suitable protocol, examples of which include RFID and NFC. This obviates the need for a battery in the interdental cleaner 10.

Embodiments of the reading module 32 include those having a power cable 38 for connection to an external power source, such as ac power from an outlet or de power received through a USB. Other embodiments include those in which the reading module 32 includes a battery.

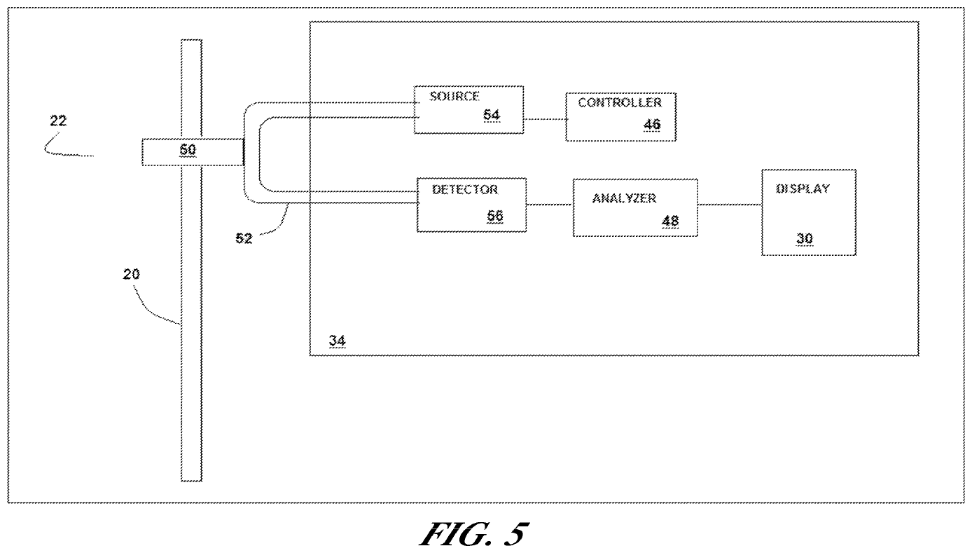

FIG. 4 shows an embodiment that features more than one functionalized sensor element 26. The different functionalized sensor elements 26 have been functionalized to selectively bind to different analytes. The plural functionalized sensor elements 26 provide corresponding signals 40, 42 to a multiplexer 44. These signals 40, 42 provide information concerning different analytes.

Based on a selection signal 44 provided by a controller 46, a selected one of the plural signals 40, 42 is provided to an analyzer 48, which outputs a relevant result on the display 30. This enables more than one analyte to be sensed from the same sample of saliva. Although only two second functionalized sensor elements 26 are shown, it should be apparent that this technique is suitable for any number of second electrodes 26.

In the foregoing example, the sensor 22 is an electrochemical sensor. In such cases, the binding of the analyte to a functionalized sensor element 26 changes its electrical properties, thereby providing a basis for sensing the analyte.

However, the complex that forms from the interaction between the analyte and a functionalized sensor element can change properties other than electrical properties. In particular, the formation of such a complex can also change optical properties. Such changes can be sensed by implementing optical sensor elements into the sensor 22 in addition to or as an alternative to electrochemical sensor elements.

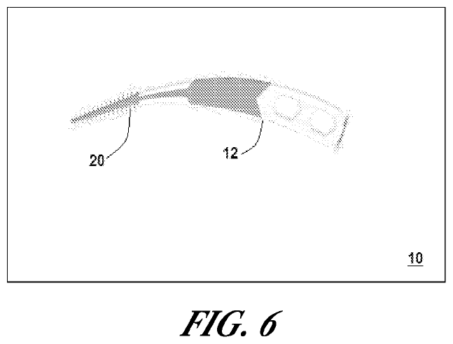

FIG. 5 shows an example of circuitry 34 used in connection with a sensor 22 having an optically-active material 50 that has been functionalized to bind to a particular analyte. In some embodiments, the active material 50 comprises a material susceptible to surface-plasmon resonance that has been functionalized with a molecule that selectively binds to the analyte of interest. Examples of such molecules include antibodies, aptamers, enzymes, nanobodies, and molecularly-imprinted polymers. Examples of materials that are susceptible to surface-plasmon resonance are gold and palladium.

An optical fiber 52 that extends from a source 54 to a detector 56 is coupled to the optically-active material 50 to enable it to interact with light carried by the fiber 52. Following activation of the source 54 by the controller 46, the detector 56 detects the result of the interaction and provides it to the analyzer 48, which then causes information about the analyte to be provided to the display 30. Similar circuitry 34 is used to sense the analyte based on Raman spectroscopy.

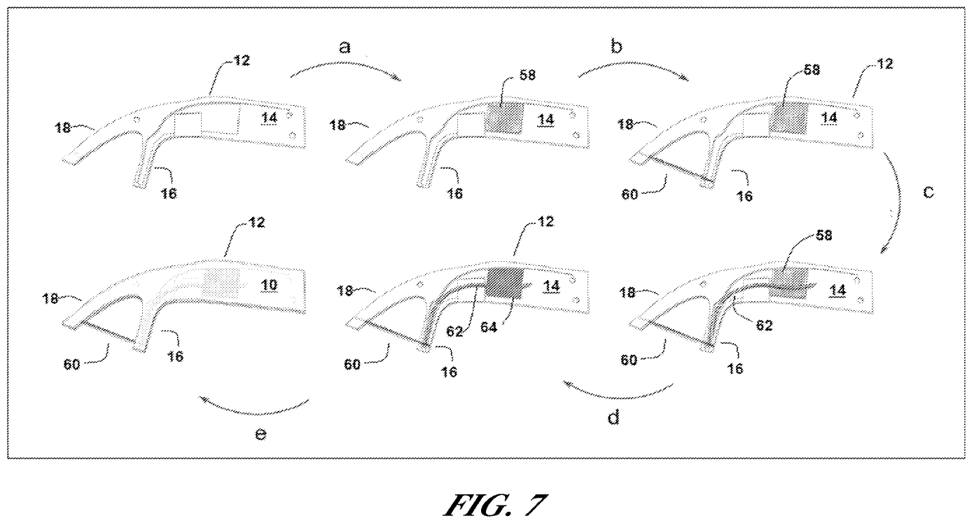

It should be apparent that the principle upon which the invention is based does not rely on the use of an interdental cleaner 10 having the form shown in FIG. 1. In particular, by having a sufficiently stiff wicking material 20, it is possible to omit the two arms 16, 18 to construct an interdental cleaner 10 that functions more as a pick than as a flosser. Such an embodiment is shown in FIG. 6.

FIG. 7 shows states in the construction of an interdental cleaner 10 of the form shown in FIG. 1.

Construction begins with constructing the upper and lower halves of the frame 14 through additive manufacturing and adding an adsorbent layer 58 to the floor of the frame 14 (step “a”). A first length 60 of wicking material 20 is then stretched across the two arms 16, 18 (step “b”).

Construction continues with adding the second length 62 of wicking material 20 and coupling a distal end to the first length 60 of the wicking material 20 and laying a proximal end of the second length 62 over the adsorbent layer 58 (step “c”). The second length 62 is then covered by an absorbent layer 64 that comprises the circuitry 34 (step “d”), after which the upper half of the frame 14 is joined to the lower half (step “e”).

Having described the invention and a preferred embodiment thereof, what is claimed as new and secured by letters patent is:

1. The apparatus of claim 22, wherein said platform comprises an interdental cleaner, said interdental cleaner comprising a handle, said wicking material, said sensor, and said sensing circuitry, wherein said sensor and said sensing circuitry are disposed in said handle, wherein said wicking material comprises a first length, which is external to said handle, and a second length, which extends into said handle to make contact with said sensor, whereby exposure of said first length to said saliva causes capillary transport of said saliva towards said second length, and wherein said sensor comprises a sensing element that is functionalized to selectively bind to said analyte in said saliva.

2. The apparatus of claim 1, further comprising a frame having first and second arms, wherein said first length extends across said arms.

3. The apparatus of claim 1, wherein said first length comprises a stiff wicking material that extends away from said handle.

4. The apparatus of claim 1, further comprising a display disposed on said handle and in communication with said sensing circuitry, wherein said display is configured to receive information from said sensing circuitry indicative of said analyte and to display said information.

5. The apparatus of claim 1, further comprising a reader that couples to said handle and reads information about said analyte from said sensing circuitry in said handle.

6. (canceled)

7. (canceled)

8. (canceled)

9. The apparatus of claim 1, wherein said sensor comprises a radiation source, a radiation detector, and a fiber extending therebetween, wherein said fiber is coupled to a material having an optical property that changes in response to presence of said analyte.

10. The apparatus of claim 1, wherein said sensor comprises a detector and a sensor element, wherein said sensor element selectively binds to said analyte, and wherein said detector senses resonance of surface plasmons on said sensor element.

11. The apparatus of claim 1, wherein said sensor comprises a sensing element and a detector, wherein said detector comprises a spectrometer that senses a lengthening of wavelength of electromagnetic radiation as a result of a loss of energy in said electromagnetic radiation due to interaction thereof with said sensor element.

12. The apparatus of claim 1, wherein said sensor element is one of a plurality of sensor elements of said sensor, each of which has been functionalized for a corresponding analyte in said saliva, wherein said circuitry comprises a multiplexer, a controller, and an analyzer, and wherein said multiplexer receives a selection signal from said controller and, in response, provides a signal from a corresponding one of said sensor elements to said analyzer.

13. The apparatus of claim 1, wherein said sensing element is functionalized by an antibody that selectively binds to said analyte.

14. The apparatus of claim 1, wherein said sensing element is functionalized by an aptamer that selectively binds to said analyte.

15. The apparatus of claim 1, wherein said sensing element is functionalized by a molecularly-imprinted polymer that selectively binds to said analyte.

16. The apparatus of claim 1, wherein said sensing element is functionalized by an enzyme that selectively binds to said analyte.

17. The apparatus of claim 1. wherein said sensing element is functionalized by a nanobody that selectively binds to said analyte.

18. The apparatus of claim 1, wherein said sensor comprises an electrode that is made from a material selected from the group consisting of carbon, graphite, and gold and an electrode that is made from screen-printed ink that comprises a material selected from the group consisting of silver, silver chloride, and platinum.

19. The apparatus of claim 1, wherein said first length of said wicking material comprises dental floss and said second length comprises a microfluidic channel coupled to said first length.

20. A method comprising exposing a first length of a wicking material of an interdental cleaner to saliva, thereby causing saliva to flow by capillary action towards a second length of said wicking material, said second length being internal to a handle of said interdental cleaner, at a sensor in said handle, sensing an interaction of an analyte in said saliva with a functionalized sensor element of said sensor, and providing information indicative of said interaction.

21. A method comprising exposing a wicking material of a platform to saliva, thereby causing said saliva to flow by capillary action, the method further comprising, at a sensor in said handle, sensing an interaction of an analyte in said saliva with a functionalized sensor element of said sensor and providing information indicative of said interaction.

22. An apparatus comprising a platform for sampling saliva via capillary transport, wicking material, a sensor, and sensing circuitry, wherein said sensor and said sensing circuitry are coupled to together, wherein said wicking material contacts said sensor, wherein said sensor comprises a sensing element that is functionalized to selectively bind to said analyte in said saliva, whereby exposure of said wicking material to said saliva causes capillary transport of said saliva towards said sensor.

23. The apparatus of claim 22, wherein said platform comprises a pick.
